(19) United States
(12) Patent Application Publication
(10) Pub. No.: US 2007/0134185 A1
(43) Pub. Date: Jun. 14, 2007

(54) Use of dithiols in a hair-perming composition

(76) Inventors: Henri Samain, Bievres (FR); Gilles Genain, Paris (FR); Aude Livoreil, Paris (FR); Gabin Vic, Venette (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 10/555,788

(22) PCT Filed: May 4, 2004
(86) PCT No.: PCT/FR04/01071

§ 371 Date: Sep. 1, 2006

Related U.S. Application Data
(60) Provisional application No. 60/477,366, filed on Jun. 11, 2003.

(30) Foreign Application Priority Data
May 6, 2003 (FR) 03/05496

(51) Int. Cl. A61K 8/58

(52) U.S. Cl. 424/70.4

(57) ABSTRACT

The subject of the invention is the use of particular dithiols in a composition for permanent deformation of hair. It also relates to a method for permanent deformation of hair using these dithiols, and cosmetic compositions containing at least one of these dithiols and at least one compound chosen from nonionic, anionic, cationic, amphoteric or zwitterionic polymers and surfactants or cosmetic compositions containing specific dithiols.

The subject of the invention is the use of particular dithiols in a composition for permanent deformation (“perming”) of hair. It also relates to a method for permanent deformation of hair using these dithiols, and cosmetic compositions containing at least one of these dithiols and at least one compound chosen from nonionic, anionic, cationic, amphoteric or zwitterionic polymers and surfactants or cosmetic compositions containing specific dithiols.

The technique for carrying out permanent deformation of hair consists, in a first instance, in opening the disulphide bonds in keratin (cystine) using a composition containing a reducing agent (reduction stage), and then after having preferably rinsed the hair, in reconstituting, in a second stage, the said disulphide bonds by applying an oxidizing composition (oxidation stage, also called fixing stage) so as to give the hair the desired shape. This technique makes it possible to carry out, without distinction, either the curling of hair, or its straightening or its relaxing, it being possible for the hair, depending on the cases, to be subjected to stress during the entire or part of the deformation process.

Compositions for carrying out the first stage of a permanent waving operation are generally provided in the form of lotions, creams, gels, powders to be diluted in a liquid carrier, and contain a reducing agent, preferably a thiol. Among the latter, those commonly used are cysteine and thioglycolic acid and its esters, in particular glyceryl monothioglycolate. Thioglycolic acid is particularly effective for reducing the disulphide bonds in keratin and may be considered as alkaline pH, in particular in the form of ammonium thioglycolate, as the reference compound in permanent waving. It has nevertheless the disadvantage of emitting an unpleasant odour. To overcome this, use is generally made of a perfume which makes it possible to mask the odours.

Cysteine produces a much weaker odour than that of thioglycolic acid, but the curliness obtained is much less and far from being satisfactory. Furthermore, cysteine requires the use of a very alkaline pH.

Glyceryl monothioglycolate is also very foul-smelling. It is, on the other hand, used at a pH close to neutral pH, but its performance is notably lower than that of thioglycolic acid.

Various studies have been carried out in order to overcome the disadvantages of these reducing agents, and to this effect, the use of novel reducing compounds or systems has been proposed. Thus, in patents U.S. Pat. No. 2,719,813 and U.S. Pat. No. 2,719,814, the use of mercaptan and its disulphide as reducing system for hair has already been proposed, recommending more particularly adjusting the pH of the reducing composition between 8.5 and 9.5. Patent U.S. Pat. No. 2,719,813 relates especially to a reducing composition for permanent waving, consisting of dithiodiglycerol and thioglycerol, and patent U.S. Pat. No. 2,719,814 a reducing composition containing in particular thiohydracrylic and dithiohydracrylic acid.

Moreover, patent U.S. Pat. No. 5 460 806 discloses the use of bis(2-mercaptoethyl)sulphone (BMS) as reducing agent in a hair process for straightening or curling hair, without specifying the pH for using reducing solutions containing such reducing agents. This compound is very sparingly soluble in water and therefore its use is practically impossible under the usual conditions for permanent deformation.

Finally, the use of certain dithiols is already known in the cosmetic field. In particular, French patent application, filed by the Applicant under the number 0204 352, and not yet published, discloses a method of metallic coating conferring, on the keratinous fibres, cosmetic properties which are stable to shampoos. According to this method, a composition containing at least one metal salt is applied to the keratinous fibres, and then a composition containing at least one reducing agent is applied. Among the latter, the following dithiols are mentioned: BMS (bismercaptoethylsulphone), DTT (dithiothreitol), DMH (N,N′dimethyl-N,N′-bis(mercaptoacetyl)hydrazine).

In this French patent application, it is stated that the keratinous fibres may be reduced prior to the application of the composition containing at least one metal salt. It is envisaged that this reduction may be carried out with one of the reducing agents described in the text of this application, such as the dithiols previously mentioned.

However, the reducing compositions for permanent waving known up until now do not yet give complete satisfaction, given that degradations of the hair fibre are too high.

In addition, when a permanent waving is applied to hair which has been subjected to dyeing, a very high stripping of the colour is observed and the surface of the hair is greatly damaged. Conversely, if a dye is applied to permanently waved hair, the colour obtained is very different from the colour normally obtained on natural non-permanently waved hair.

The problem posed by the invention is to provide a reducing composition, for permanent deformation of hair, which is more effective than those which already exist, in particular in terms of degree, of vitality or of quality of curl, while reducing damage to the hair, it being necessary for this reducing composition, additionally, to be practically without effect on an artificial coloration of the keratinous fibres.

To solve this problem, the invention proposes the use, in order to reduce the disulphide bonds in keratin, in a reducing composition for deformation of the keratinous fibre, at a pH of between 5 and 8.5, of dithiols of formula I, having a solubility in water greater than 0.1 M:
HS-A-SH (I)

A is a saturated or unsaturated, linear or cyclic, C3 to C10 hydrocarbon chain, this chain optionally carrying one or more C1 to C4 branches and being capable of being interrupted by one or more heteroatoms such as sulphur, oxygen, nitrogen, silicon or phosphorus and being also capable of comprising one or more substituents, such as hydroxyl, amine, carbamate, carbonate, hydrazine, ether, acid, ester, amide, cyano or ureido groups.

The choice of the pH in the interval between 5 and 8.5 leads to the following advantages: low degradation of the fibre and better cosmetic result compared with the use of the same dithiols at a more alkaline pH.

The solubility in water of the dithiols of formula I is measured at pH=7 and at a temperature of 30° C.

The expression “permanent deformation” is understood to mean the permanent curling (permanent waving), the straightening or the relaxing of hair.

Preferably, A is a hydrocarbon chain comprising from 3 to 8 carbon atoms, and more preferably from 4 to 6 carbon atoms.

Advantageously, the pH of the reducing composition comprising the dithiols of formula I is between 5.5 and 8.5, and more preferably between 6 and 8.

Preferably, the pKa, at 25° C., of the dithiol of formula I is less than 9.

Another subject of the invention relates to a method for the permanent deformation of hair using a reducing composition comprising at least one compound of formula (I).

The compounds of formula (I) are generally prepared according to the procedures described in the following references:
J. Org. Chem 91, vol 56, p 2332;
J. Org. Chem 91, vol 56, p 7328;
Methods in Enzymology, 1995, vol 251, p 167.

Among the preferred compounds of general formula (I), there may be mentioned in particular the compounds indicated below: 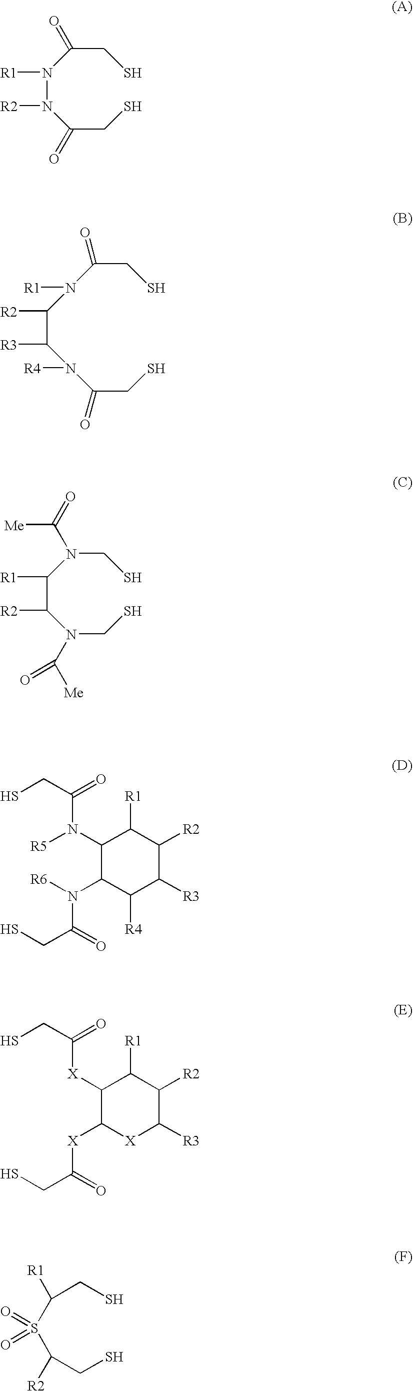 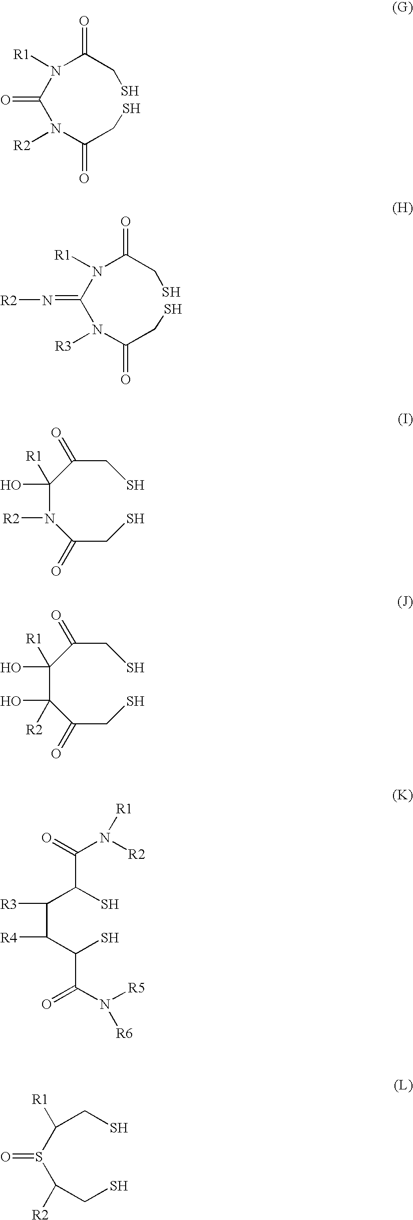
With R1, R2, R3, R4, R5 and R6 representing, independently of each other: H, OH, COOH, NH₂, a linear or branched C1 to C10 alkyl radical, a linear or branched C2 to C10 alkenyl radical, a linear or branched C2 to C10 alkynyl radical, it being possible for these radicals to be interrupted by one or more heteroatoms such as sulphur, oxygen, nitrogen, silicon or phosphorus and it being possible also for these radicals to comprise one or more substituents such as hydroxyl, amine, carbamate, carbonate, hydrazine, ether, acid, ester, amide, cyano or ureido groups; X═O, S or NR1.

Most particularly preferred are the compounds of formula I below and the corresponding optical isomers, where appropriate:

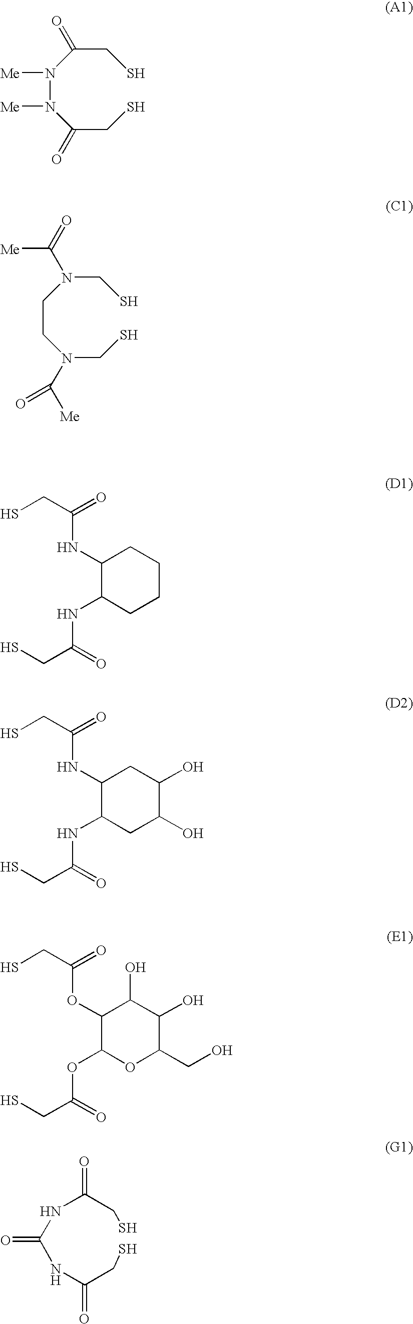

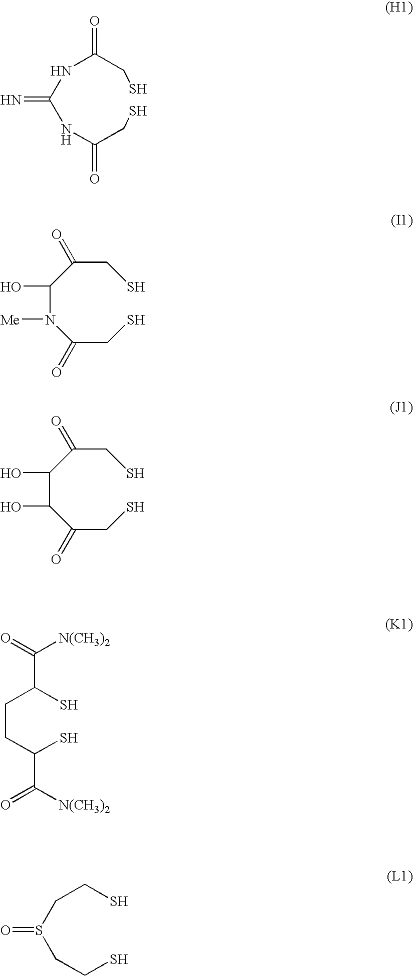

Most particularly preferable are the following compounds of formula I: A1, D1 and K1 and the corresponding optical isomers.

The method in accordance with the invention for permanent deformation of hair comprises the application of a reducing composition comprising, as reducing agent, a compound of formula (I). The hair is shaped using, for example, mechanical means well known to persons skilled in the art, such as curlers, the reducing composition being applied before or after the optional means for shaping the hair, and a fixing composition being applied after the reducing composition, with or without intermediate or subsequent stage for rinsing or applying an intermediate composition.

According to the present invention, the permanent deformation of hair preferably consists, in a first stage, in reducing the disulphide bonds in keratin by applying, for about 5 to 60 min, a reducing composition as defined above and then, in a second stage, in reforming the said bonds by applying an oxidizing composition or optionally allowing atmospheric oxygen to act.

Preferably, in the case of permanent waving, a reducing composition as defined above is applied to wet hair curled beforehand on rollers having a diameter of 4 to 20 mm, it being possible for the composition to be optionally applied as the hair is curled; the reducing composition is then allowed to act for a period of 5 to 60 minutes, preferably of 5 to 30 minutes, and then abundantly rinsed; after which an oxidizing composition which makes it possible to reform the disulphide bonds in keratin is applied to the curled hair for a leave-in time of 2 to 10 minutes. After having removed the rollers, the hair is abundantly rinsed.

The reducing composition may be left in under heat.

The oxidizing composition is of the type commonly used and contains, for example, as oxidizing agent, hydrogen peroxide, an alkali metal bromate, a persalt, a polythionate or an alkali metal bromate and persalt mixture. The concentration of hydrogen peroxide may vary from 1 to 20 volumes and preferably from 1 to 10, the concentration of alkali metal bromate from 2 to 12% and that of persalt from 0.1 to 15% by weight relative to the total weight of the oxidizing composition. The pH of the oxidizing composition is generally between 2 and 10. This oxidation may be carried out immediately or may be carried out later.

The deformation of hair according to the invention may also consist in a method for straightening or relaxing hair, in which a reducing composition according to the invention is applied to the hair, and then the hair is subjected to mechanical deformation which makes it possible to fix it in its new shape, by an operation for smoothing hair with a wide-toothed comb, with the back of a comb or with the hand. After a leave-in time of 5 to 60 minutes, in particular of 5 to 30 minutes, another smoothing is carried out, and then careful rinsing is carried out and an oxidizing or fixing composition as defined above is applied, and it is allowed to act for about 2 to 10 minutes, and then the hair is abundantly rinsed.

In the permanent waving compositions according to the invention, the reducing agent of general formula (I) is normally present at a concentration of between 0.05 and 30%, and preferably between 0.1 and 20% by weight relative to the total weight of the reducing composition.

The pH is obtained with the aid of an alkaline agent such as for example aqueous ammonia, monoethanolamine, diethanolamine, triethanolamine, 1,3-propanediamine, an alkali metal or ammonium carbonate or bicarbonate, an organic carbonate such as guanidine carbonate, an alkali metal hydroxide or using an acidifying agent such as for example hydrochloric acid, acetic acid, lactic acid, oxalic acid or boric acid.

The reducing composition may also contain, in combination, another known reducing agent, such as for example thioglycolic acid, glyceryl or glycol monothioglycolate, cysteamine and its C1-C4 acyl derivatives such as N-acetylcysteamine or N-propionyl-cysteamine, cysteine, N-acetylcysteine, N-mercaptoalkylamides of sugars such as N-(2-mercaptoethyl)gluconamide, beta-mercaptopropionic acid and its derivatives, thiolactic acid and its esters such as glyceryl monothiolactate, thiomalic acid, pantetheine, thioglycerol, alkali or alkaline-earth metal sulphites or bisulphites, the N-(mercaptoalkyl)-ω-hydroxyalkylamides described in patent application EP 354 835 and the N-mono- or N,N-dialkylmercapto-4-butyramides described in patent application EP 368763, the aminomercaptoalkylamides described in patent application EP 403 267 and the alkylaminomercapto-alkylamides described in patent application EP 432 000.

According to a preferred embodiment, the reducing composition also contains a surfactant of the nonionic, anionic, cationic or amphoteric type, and among these, there may be mentioned alkyl sulphates, alkyl benzenesulphates, alkyl ether sulphates, alkyl sulphonates, quaternary ammonium salts, alkylbetaines, oxyethyleneated alkylphenols, alkanolamides of fatty acids, oxyethyleneated fatty acid esters and other nonionic surfactants of the hydroxypropyl ether type.

When the reducing composition-contains at least one surfactant, the latter is generally present at a maximum concentration of 30% by weight, preferably between 0.5 and 10% by weight relative to the total weight of the reducing composition.

With the aim of improving the cosmetic properties of the hair and also to attenuate or avoid its degradation, the reducing composition may also contain a treatment agent of the cationic, anionic, nonionic or amphoteric type.

Among the particularly preferred treatment agents, there may be mentioned in particular those described in French patents No. 2,598,613 and No. 2,470,596. It is also possible to use, as treatment agents, volatile or non-volatile, linear or cyclic, silicones and mixtures thereof, polydimethylsiloxanes, quaternized polyorganosiloxanes such as those described in French patent application No. 2,535,730, polyorganosiloxanes with aminoalkyl groups modified with alkoxycarbonylalkyl groups such as those described in U.S. Pat. No. 4,749,732, polyorganosiloxanes such as the polydimethylsiloxane-polyoxyalkyl copolymer of the Dimethicone Copolyol type, a polydimethylsiloxane with terminal stearoxy groups (stearoxydimethicone), a polydimethylsiloxane-dialkylammonium acetate copolymer or a polydimethylsiloxane-polyalkylbetaine copolymer described in British patent No. 2,197,352, polysiloxanes organomodified with mercapto or mercaptoalkyl groups such as those described in French patent No. 1,530,369 and in European patent application No. 295,780, and silanes such as stearoxytrimethyl-silane.

The reducing composition may also contain other treatment ingredients such as cationic polymers such as those used in the compositions of French patents No. 79,32078 (U.S. Pat. No. 2,472,382) and 80,26421 (U.S. Pat. No. 2,495,931), or cationic polymers of the ionene type such as those used in the compositions of the Luxembourg patent No. 83703, basic amino acids (such as lysine, arginine) or acidic amino acids (such as glutamic acid, aspartic acid), peptides and their derivatives, protein hydrolysates, waxes, swelling and penetrating agents or agents which make it possible to enhance the efficacy of the reducing agent such as the SiO2/PDMS (polydimethylsiloxane) mixture, dimethylisosorbitol, urea and its derivatives, pyrrolidone, N-alkylpyrrolidones, thiamorpholinone, alkylene glycol or dialkylene glycol alkyl ethers such as for example propylene glycol monomethyl ether, dipropylene glycol monomethyl ether, ethylene glycol monoethyl ether and diethylene glycol monoethyl ether, C3-C6 alkanediols such as for example 1,2-propanediol and 1,2-butanediol, 2-imidazolidinone and other compounds such as fatty alcohols, lanolin derivatives, active ingredients such as pantothenic acid, anti-hair loss agents, antidandruff agents, thickeners, suspending agents, sequestering agents, opacifying agents, colorants, sunscreens and perfumes and preservatives.

The reducing composition according to the invention is mainly provided in aqueous form, in particular in the form of a thickened or non-thickened lotion, a cream or a gel.

The reducing composition according to the invention may also be of the exothermic type, that is to say causing some heating during application to the hair, which is pleasant for the person undergoing the first stage of permanent waving or hair straightening.

The reducing composition according to the invention may also contain a solvent such as for example ethanol, propanol or isopropanol, or alternatively glycerol, at a maximum concentration of 20% relative to the total weight of the composition.

The vehicle of the compositions according to the invention is preferably water or an aqueous-alcoholic solution of a lower alcohol such as ethanol, isopropanol or butanol.

When the compositions are intended for a hair straightening or relaxing operation, the reducing composition is preferably in the form of a thickened cream so as to maintain the hair as straight as possible. These creams are produced in the form of “heavy” emulsions, for example based on glyceryl stearate, glycol stearate, self-emulsifiable waxes or fatty alcohols.

It is also possible to use liquids or gels containing thickening agents such as carboxyvinyl polymers or copolymers which “stick” the hair and keep it in the smooth position during the leave-in time.

The invention also relates to cosmetic compositions comprising, in a cosmetically acceptable medium, at least one dithiol of formula I chosen from the compounds of formulae (B), (C), (E), (F), (G), (H), (I), (J), (L), (M) and (N). (M) and (N) have the formulae below:

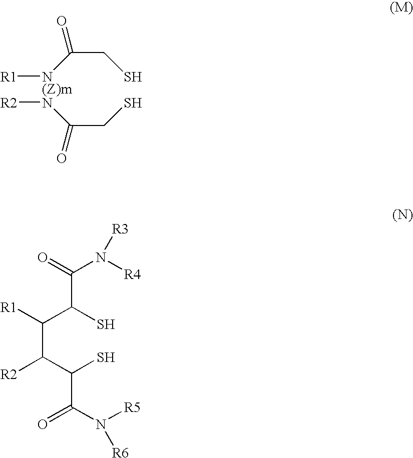

in which m denotes 0 or 1.

R1 and R2 represent, independently of each other, H, OH, COOH, NH2, a linear or branched C2 to C10 alkyl radical, a linear C2 to C10 alkenyl radical, a linear or branched C2 to C10 alkyl radical, it being possible for these radicals to be interrupted by one or more heteroatoms such as sulphur, oxygen, nitrogen, silicon or phosphorus, and it being possible for these radicals to comprise one or more substituents, such as hydroxyl, amine, carbamate, carbonate, hydrazine, ether, ester, amide, cyano or ureido groups; with the restriction that,

if m=1, R1 and R2 represent, independently of each other, a methyl radical or a radical R1 as indicated above,

Z represents a bond belonging to a C6 carbocycle substituted with one or more groups Ri, which are identical or different, Ri denoting a methyl radical or a radical R1, as indicated above other than hydrogen or a bond belonging to an unsubstituted C6 carbocycle if at least one of R1 or R2 is different from a hydrogen atom,

R3, R4, R5 and R6 have, independently of each other, the same meanings as R1 and R2 as indicated above, and may also denote a methyl radical, if R1 and R2 do not both denote a hydrogen atom or if one of them is at least different from a methyl radical.

The invention finally relates to cosmetic compositions containing at least one compound of formula I as defined above and at least one compound, chosen from surfactants and nonionic, anionic, cationic, amphoteric or zwitterionic polymers. All these compositions additionally contain the additives mentioned above.

The invention also relates to a kit, in particular for the permanent deformation of hair comprising, in a first compartment, as reducing composition, a composition in accordance with the invention comprising a compound of formula (I), and in a second compartment, an oxidizing composition.

The invention will be understood more clearly with the aid of the nonlimiting example which follows and which constitutes a preferred embodiment of the compositions according to the invention.

### EXAMPLES

A reducing lotion is prepared:

| | |
|---|---|
| Compound A1 | 20.83 g |
| Aqueous ammonia | qs pH = 7 |
| Water | qs 100 g |

The above lotion is applied to locks of grey hair which is 90% white. Curls are produced using curlers. The lotion is allowed to act on the curled hair for 15 minutes. The hair is rinsed with water. A conventional fixing composition based on hydrogen peroxide (hydrogen peroxide at 8 volumes, pH=3), is applied. Another rinsing is carried out. The curlers are removed.

Good curliness is observed. If a 7.44 CRESCENDO® oxidation dye is applied to hair permanently waved with a compound Al as indicated above, a colour is observed which is closer to that obtained on non-permanently waved hair than in the case where a permanent waving treatment identical to the preceding one is used, replacing A1 with thioglycolic acid, under production conditions allowing equivalent curliness to be obtained.

1. Use, in order to reduce the disulphide bonds in keratin, in a reducing composition for deformation of the keratinous fibre, at a pH of between 5 and 8.5, of dithiols of formula I, having a solubility in water greater than 0.1 M:
HS-A-SH (I)
A is a saturated or unsaturated, linear or cyclic, C3 to C10 hydrocarbon chain, this chain optionally carrying one or more C1 to C4 branches and being capable of being interrupted by one or more heteroatoms such as sulphur, oxygen, nitrogen, silicon or phosphorus and being also capable of comprising one or more substituents, such as hydroxyl, amine, carbamate, carbonate, hydrazine, ether, acid, ester, amide, cyano or ureido groups.

2. Use according to claim 1, characterized in that the pH of the reducing composition comprising the dithiols of formula I is between 5.5 and 8.5, and more preferably between 6 and 8.

3. Use according to claim 1 or 2, characterized in that A is a hydrocarbon chain comprising from 3 to 8 carbon atoms, and more preferably from 4 to 6 carbon atoms.

4. Use according to any one of the preceding claims, characterized in that the pKa, at 25° C., of the dithiol of formula I is less than 9.

5. Use according to any one of the preceding claims, characterized in that the dithiol of formula (I) corresponds to one of the following formulae: 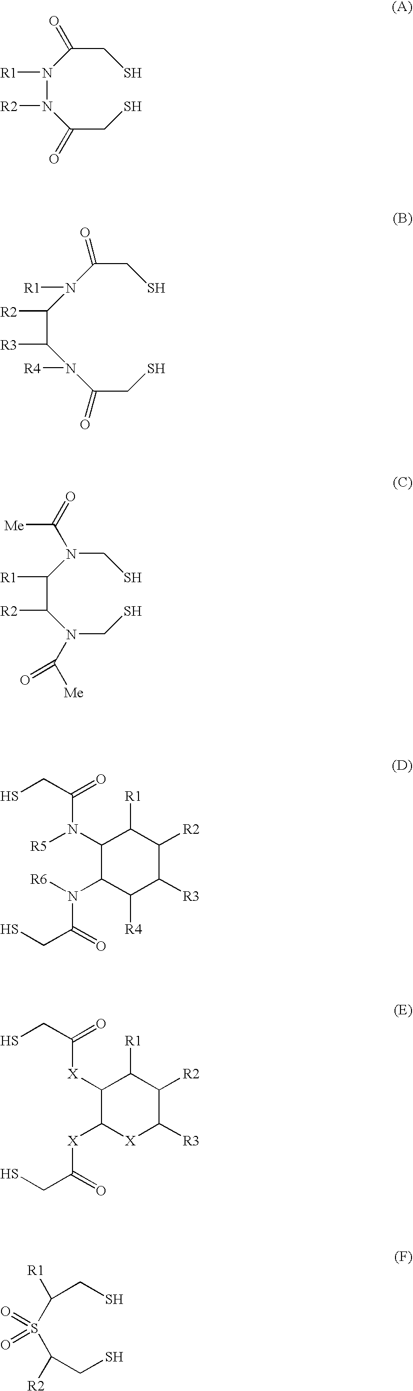 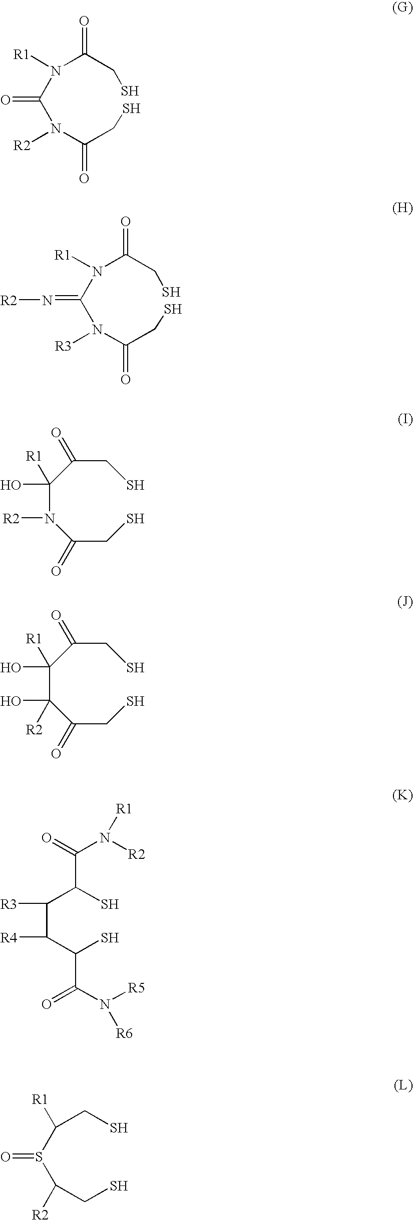
With R1, R2, R3, R4, R5 and R6 representing, independently of each other: H, OH, COOH, NH₂, a linear or branched C1 to C10 alkyl radical, a linear or branched C2 to C10 alkenyl radical, a linear or branched C2 to C10 alkynyl radical, it being possible for these radicals to be interrupted by one or more heteroatoms such as sulphur, oxygen, nitrogen, silicon or phosphorus and it being possible also for these radicals to comprise one or more substituents such as hydroxyl, amine, carbamate, carbonate, hydrazine, ether, acid, ester, amide, cyano or ureido groups; X═O, S or NR1.

6. Use according to any one of the preceding claims, characterized in that the dithiol of formula I corresponds to one of the following formulae, the corresponding optical isomers, where appropriate:

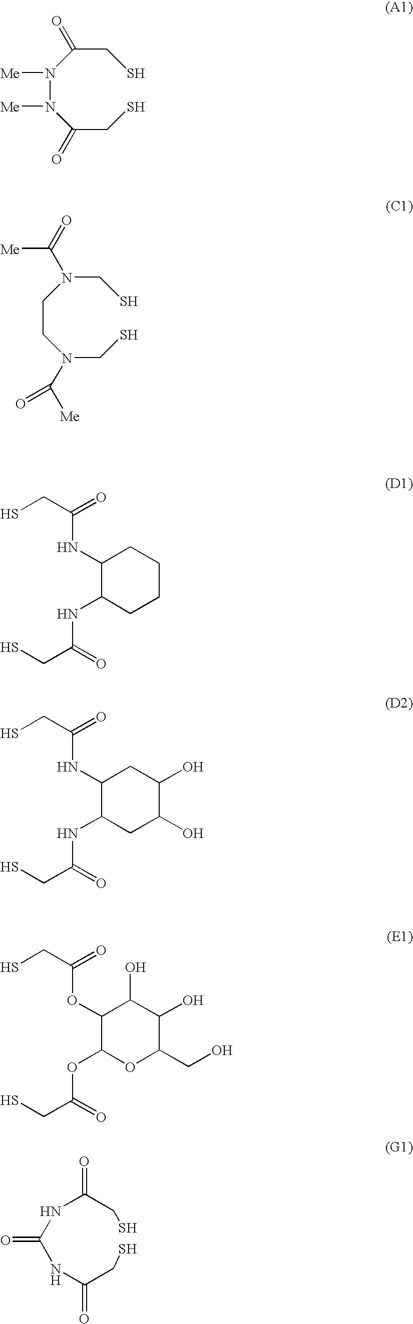

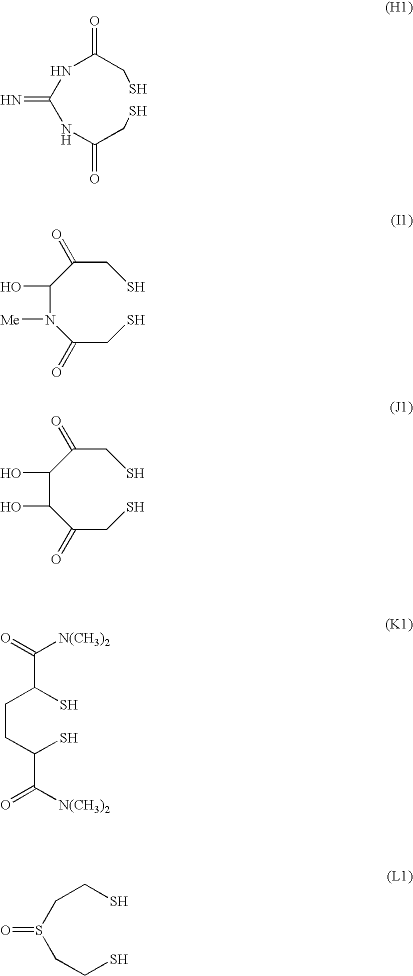

7. Use according to claim 6, characterized in that the compounds of formula I are the following: A1, D1 and K1 and the corresponding optical isomers.

8. Use according to any one of the preceding claims, characterized in that the reducing agent of general formula (I) is present at a concentration of between 0.05 and 30%, and preferably between 0.1 and 20% by weight relative to the total weight of the reducing composition.

9. Use according to any one of the preceding claims, characterized in that the composition additionally contains an additive chosen from another known reducing agent, a surfactant of the nonionic, anionic, cationic or amphoteric type, a treatment agent of the cationic, anionic, nonionic or amphoteric type, fatty alcohols, lanolin derivatives, active ingredients such as pantothenic acid, anti-hair loss agents, antidandruff agents, thickeners, suspending agents, sequestering agents, opacifying agents, colorants, sunscreens and perfumes and preservatives.

10. Method for permanent deformation of hair, characterized in that it comprises the application of a reducing composition defined in any one of claims 1 to 8 containing, as reducing agent, a compound of formula (I), the hair being shaped by appropriate means; a fixing composition being additionally applied after the reducing composition, after optional rinsing.

11. Cosmetic composition comprising, in a cosmetically acceptable medium, at least one dithiol of formula (I) chosen from the compounds of formulae (B), (C), (E), (F), (G), (H), (I), (J) and (L) according to claim 4 and the compounds of the following formulae (M) and (N): 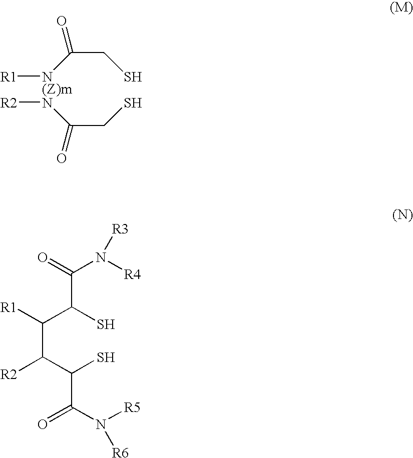
in which m denotes 0 or 1.
R1 and R2 represent, independently of each other, H, OH, COOH, NH2, a linear or branched C2 to C10 alkyl radical, a linear C2 to C10 alkenyl radical, a linear or branched C2 to C10 alkyl radical, it being possible for these radicals to be interrupted by one or more heteroatoms such as sulphur, oxygen, nitrogen, silicon or phosphorus, and it being possible for these radicals to comprise one or more substituents, such as hydroxyl, amine, carbamate, carbonate, hydrazine, ether, ester, amide, cyano or ureido groups; with the restriction that,
if m=1, R1 and R2 represent, independently of each other, a methyl radical or a radical R1 as indicated above,
Z represents a bond belonging to a C6 carbocycle substituted with one or more groups Ri, which are identical-or different, Ri denoting a methyl radical or a radical R1, as indicated above other than hydrogen or a bond belonging to an unsubstituted C6 carbocycle if at least one of R1 or R2 is different from a hydrogen atom,
R3, R4, R5 and R6 have, independently of each other, the same meanings as R1 and R2 as indicated above, and may also denote a methyl radical, if R1 and R2 do not both denote a hydrogen atom or if one of them is at least different from a methyl radical.

12. Cosmetic compositions containing at least one compound of formula I and at least one compound chosen from nonionic, anionic, cationic, amphoteric or zwitterionic polymers and surfactants,
HS-A-SH (I)
A is a saturated or unsaturated, linear or cyclic, C3 to C10 hydrocarbon chain, this chain optionally carrying one or more C1 to C4 branches and being capable of being interrupted by one or more heteroatoms such as sulphur, oxygen, nitrogen, silicon or phosphorus and being also capable of comprising one or more substituents, such as hydroxyl, amine, carbamate, carbonate, hydrazine, ether, acid, ester, amide, cyano or ureido groups.
